(19) United States
(12) Patent Application Publication
(10) Pub. No.: US 2013/0320574 A1
(43) Pub. Date: Dec. 5, 2013

(54) AERODYNAMIC FORMULA DISPERSING APPARATUS

(71) Applicant: The Yankee Candle Company, Inc., South Deerfield, MA (US)

(72) Inventors: Jonathan SICKINGER, South Hadley, MA (US); Cheriyan B. THOMAS, Farmington, CT (US); John E. CAGLE, South Deerfield, MA (US); Thomas J. KENNEDY, III, Wilbraham, MA (US)

(73) Assignee: THE YANKEE CANDLE COMPANY, INC., South Deerfield, MA (US)

(21) Appl. No.: 13/896,835

(22) Filed: May 17, 2013

Related U.S. Application Data
(60) Provisional application No. 61/648,686, filed on May 18, 2012.

(51) Int. Cl. A61L 9/03; A61L 9/12

(52) U.S. Cl. CPC A61L 9/032; A61L 9/122
USPC 261/141; 261/30

(57) ABSTRACT

A formula dispersing apparatus that increases the throw, volume and efficiency of formula distribution through aerodynamic surfaces is described. The shape of the ring airfoil in the utilization of the Coandà effect allows for efficient distribution of a formula or formula mixture into a defined space.

### RELATED APPLICATIONS

This application claims priority to U.S. Provisional Application No. 61/648,686 filed on May 18, 2012, which is incorporated herein by reference.

### BACKGROUND OF INVENTION

Traditionally, devices used to disperse a volatalizable formula such as a fragrance, insect control active or medicinal formula have either relied on the passive movement of ambient air to distribute the entrained formula throughout the living space where the device is placed or on the lower vapor pressure of the formula and formula carrier through heating of the formula containing material, typically through the use of an electrical wall socket and a resistive heater.

In the case of a passive formula dispersing apparatus, the formula to be dispersed is mixed into a solid or gel matrix that slowly releases the formula to the air as the volatile materials in the solid or gel evaporate into the ambient air of the room.

In the case of a heated formula dispersing apparatus, a gel or liquid with an incorporated formula is heated to distribute the formula by volatilizing the formula and the carrier material within which the formula is incorporated.

Recently, auxiliary devices, such as a bladed fan, have been added to both the ambient and heated air formula dispersing apparatus to improve the distribution of the formula. The effectiveness, however, of the auxiliary devices, such as a fan, is low.

It is therefore an object of the present invention to provide a formula dispersing apparatus that improves the distribution of the formula.

### SUMMARY OF THE INVENTION

The embodiments of the formula dispersing apparatus described herein use a generally circular airfoil or duct to promote the diffusion or broadcast of a formula from a formula dispersing apparatus. The generally circular airfoil has the suction side of the airfoil on the inside (closest to the wall) and the pressure side on the outside. This arrangement sets up a circular flow of an air stream through the ring airfoil. The formula dispersing apparatus may also contain a circulation fan that pushes air into the circular airfoil where the fan is disposed within the circular airfoil structure.

The formula dispersing apparatus may also be comprised of an air channel within the airfoil where the air is entrained with the formula from a formula repository and then subsequently flowed out onto the edge of the airfoil. The airfoil will have a slot or slots in it such that the Coand\check{a} effect causes the formula entrained in the air stream to flow out and over the circular airfoil, enhancing the dispersion of the formula in the environment where the apparatus is placed. The circular airfoil preferably has a constant camber.

The air freshener apparatus may also include an external source of formula that is entrained into the airflow subsequent to the airflow exiting the circular airfoil.

In another embodiment, the vapor pressure of the formula mixture may be manipulated so as to increase the evaporation rate and increase the vapor pressure such that larger amounts of formula will be entrained into the air stream.

In certain embodiments a heater is used to heat the material which contains the formula in order to assist, increase, or enable the volatilization of essential oils, formulas, or other materials intended to be delivered from a porous media in the formula source to the airstream. A fan or impeller may also be used to push the formula into the airstream travelling through the airfoil.

### BRIEF DESCRIPTION OF THE DRAWINGS

FIG. 1 is a perspective view of an air freshener apparatus with a ring airfoil and a prop.

FIG. 2 is a perspective view of an alternate embodiment of an air freshener apparatus.

FIG. 3 is another perspective view of an alternate embodiment of an air freshener apparatus without a prop and with a circular airfoil and an inner distribution ring.

FIG. 4 is a cross sectional diagram of the operation of a circular airfoil.

FIG. 5 is a perspective view of the air freshener with a circular airfoil and a central nozzle.

FIG. 6 is a perspective view of an alternate embodiment of the present invention plugged into a wall.

FIG. 7 is a front view of the air freshener shown in FIG. 6.

FIG. 8 is a rear view of the air freshener shown in FIG. 6.

FIG. 9 is a cross sectional view of the air freshener of FIG. 6 taken along line 9-9 of FIG. 7.

FIG. 10 is an exploded view of the air freshener shown in FIG. 6.

FIG. 11 is a depiction of the scalar airflow through a circular airfoil.

FIG. 12 is a diagram showing the Coand\check{a} affect airflow across the interior or suction side of the circular airfoil.

FIG. 13 is a diagram of a NACA 6412 airfoil.

FIG. 14 is a diagram of a NACA airfoil measurement scheme.

### DETAILED DESCRIPTION OF THE PREFERRED EMBODIMENTS

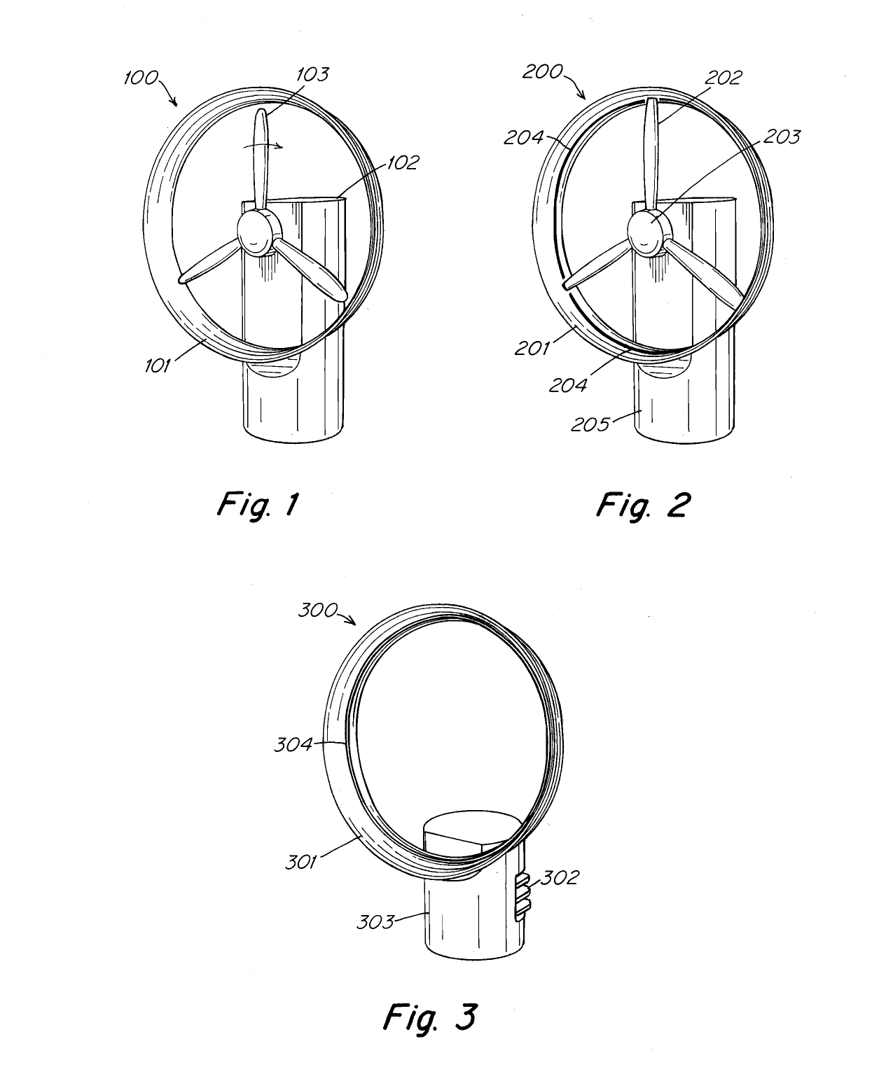

The embodiment of the formula dispersing apparatus shown in FIG. 1 depicts an formula dispersing assembly 100 that has a ring airfoil 101 and a body 102. In this assembly, the external prop 103 supplies the moving air to disperse the formula and is freewheeling. The distributed formula is also contained in the body 102 before passing through the fan. The formula is thus entrained into the air stream that is distributed through the ring airfoil 101.

In the embodiment of the formula dispersing apparatus shown in FIG. 2, the formula dispersing apparatus 200 has fixed structures 202 that attach directly to the ring airfoil 201. Air and formula communicate from the base 205 though the distribution hub 203 to the ring airfoil 201. The formula and air are then ejected through slots 204 in the ring airfoil 201 to the room environment.

FIG. 3 shows an embodiment of the formula dispersing apparatus with formula dispersing assembly 300 with just a ring airfoil 301 and a base 303. The formula dispersing body contains a means for moving air, such as a bladed fan, intake slots 302 on the base 303 and a means of supplying the air flow from the fan to the ring airfoil such that the Coand\check{a} effect is achieved. A device such as an inner ring 304 may be used to generate a jet of air and formula that may be distributed through the room through the ring airfoil 301. The ring airfoil 301 may also have slots in it where the air and entrained formula flow into the room. The slots 302 in the base of the formula dispersing apparatus 300 allow for air to flow into the base 303 and out through the inner ring 304 or slots in the ring airfoil 301. The ring airfoil 301 may also comprise a formula infused material such as a formula infused ethylene vinyl acetate (EVA) that allows the formula to escape over time and be entrained in the air flow through the ring airfoil 301.

The Coand\check{a} effect is utilized in aircraft. With the use of flaps and a “jet” sheet of air blowing over the curved surface, air moving over the wing can be “bent down” towards the ground. The flow is accelerated, due to Bernoulli's principle, and pressure is decreased. When pressure is decreased, aerodynamic lift is increased.

By decreasing the pressure, more air may be pumped through the airfoil. With the entrainment of formula into the air stream, the distribution of the formula will be greater.

All of the flow characteristics are related to Bernoulli's principle and derivations thereof

Bernoulli's principle may be broadly defined by Equation 1.

$$\frac{{v}^{2}}{2} + gz + \frac{p}{ρ} = constant \qquad Equation 1$$

v is the fluid flow speed at a point on a streamline
g is the acceleration due to gravity
z is the elevation of the point above a reference plane
p is the pressure at the chosen point
ρ is the density of the fluid at all points in the fluid

The embodiment may also be comprised of a diffuser where the Venturi effect is utilized.

The shape of the airfoil is of great importance for the distribution of the formula that is entrained into the airflow into the defined space where the air freshener is working One area of airfoil definitions are the NACA airfoils developed by the National Advisory Committee for Aeronautics (NACA) as possible shapes for wings for aircraft. The shape of the airfoil is described numerically using various parameters of the airfoil with digits following the NACA designation. For example, a NACA 6412 as 6% camber relative to the maximum cord, 4/10 of the cord for the location of the maximum camber and 12% as a maximum thickness as a percentage of the cord. The numerical code for each NACA airfoil can be entered into equations to precisely generate the cross-section of the airfoil and calculate its properties.

The equations for calculations of the various aspects of an NACA airfoil are shown in equation 2. The various aspects of the airfoil may be manipulated to increase or decrease the effect of the airfoil on airflow. For instance, a more aggressive camber will allow for greater airflow but also suffers from the separation of the airflow from the airfoil, thus lowering the efficiency of the system.

The attack angle or the angle at which the airfoil is turned also has an effect on the airflow. In this case, an airfoil that is perpendicular to the ground will have less of an effect on airflow than will an airfoil the higher degree of angle of attack. However, by changing the angle of attack, the airfoil can also possibly suffer from separation of the air stream and thus the stalling of the airflow over the airfoil.

The use of the Coand\check{a} effect will allow not only improved distribution of the airflow entrained formula into a defined space but will also decrease the separation of the airflow from aggressively cambered and angle of attack airfoils.

Equation 2
NACA four digit series airfoils

```
The NACA 4-digit series is defined by four digits, e.g.,
NACA 8412: m = 8%, p = 4/10, t = 12%
The equations are:
yₜ = (t/0.2) (0.2969√x − 0.126x − 0.3516x² + 0.2843x³ − 0.1015x⁴)
y_{c} = (m/p²) (2px − x²); for x ≦ p
y_{c} = (m/(1 − p)²) ((1 − 2p) + 2px − x²); for x > p
Where t is the maximum thickness as a percentage of the
chord, m is the maximum camber as a percentage of the
chord, p is the chordwise position of the maximum camber
as a tenth of the chord.
The final coordinates for the airfoil upper surface (x_{U}, y_{U})
and lower surface (x_{L}, y_{L}) are given by:
              x_{U} = x − yₜ(sinθ)
              y_{U} = y_{c} + yₜ(cosθ)
              x_{L} = x + yₜ(sinθ)
              y_{L} = y_{c} − yₜ(cosθ)
          where θ = arctan(Δy_{c}/Δx)
```

The NACA four digit series is one description of how to build an airfoil. There are various other ways and other NACA designations for airfoil shapes.

Throughout all these constructions, however, it is important to ensure that the airfoil operates correctly within the realm of the air flow for which is intended. As mentioned above, if the design is too aggressive, the airfoil will see separation of the airflow from the airfoil surface and thus cause stalling of the airflow.

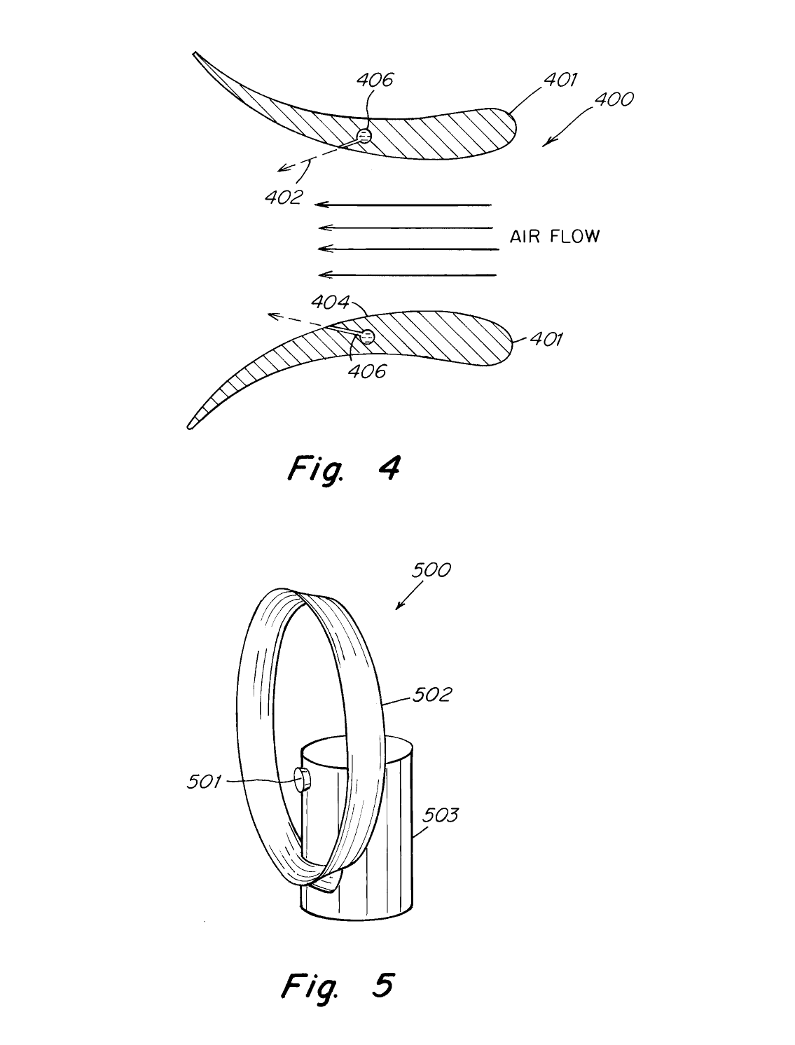

FIG. 4 shows a cross section of a circular airfoil 400 and the air flow through the airfoil 400. The cross section 401 shows an airfoil with a high camber. The suction side of the airfoil 400 is on the inside and causes a strong circulation of the air around the circular airfoil 400. Formula is emitted as a jet 402 from a slot 406 in the circular airfoil 401.

The formula dispersing apparatus 500 shown in FIG. 5 includes a nozzle 501, a circular airfoil 502 and a body 503. The air flows out through the nozzle 501 which acts like a jet to distribute the entrained formula through the ring airfoil 502. The body 503 of the air freshener 500 contains the formula and mechanism for forcing air through the jet nozzle 501.

In all of these embodiments, it is important that the formula be dispersed evenly so that an aerosol cloud is not formed which could cause an overwhelming reaction to the formula. An understanding of the inherent tendency of an ingredient to escape into the gas phase is a useful starting point when considering formula volatility. The relative molecular mass (RMM) and the boiling point of a formula ingredient will provide some guidelines to the behavior of the material. For materials whose boiling points are not known, it is generally a sound alternative to look at chromatographic behavior. For example, the retention time for a material to elute through a gas chromatographic column containing a nonpolar phase is often strongly related to the boiling point (in fact, such columns are commonly referred to as “boiling point” columns).

It is therefore important to match the Relative Molecular Mass (RMM), which can be considered as another characterization of the volatility of the formula, and the gas chromatographic characteristics of the formula mixture to the airflow through the ring airfoil. If the formula mixture vapor pressure at the operating temperature is too high and the airflow through the ring airfoil is also too high, an undesirable concentration of formula may result.

The total amount of air that is set in motion in an airfoil device receives three contributions, so to speak: (a) from the air that goes through the formula dispersing apparatus, (b) from the air that gets drawn to the suction side of the ring of the formula dispersing apparatus, and (c) from the air that gets entrained into the free shear-layer that comes out of the formula dispersing apparatus ring.

For example, an injected air/formula value of 2.5 cfm (contribution (a)) corresponds a total of 9.4 cfm of air passing through the ring (contributions (a) plus (b), above) and to a total of 267 cfm of air moving at the station 2 m downstream (i.e., contributions (a)+(b)+(c)). This example is for half of the airflow stream. Therefore the total of the airflow stream will be greater than 500 cfm at 2 m downstream from the device. If the vapor pressure of the formula is too high, say 200 kPa, the dispersion of the formula may be too high.

Is therefore desirable to have the mass flow at 2 m downstream of the device to be less than 750 cfm and the vapor pressure of the formula mixture to be less than 200 kPa. The 750 ft.³ per minute (CFM) is achieved through adjustment of (a) the speed of the fan, (b) the shape of the ring airfoil and (c) the attack angle of the ring airfoil. The vapor pressure of the formula mixture is a factor of the temperature of the formula mixture and its inherent evaporative properties. For instance, acetone has a vapor pressure of 240 hPa at 20° C. By heating the acetone, the vapor pressure would increase above 240 hPa

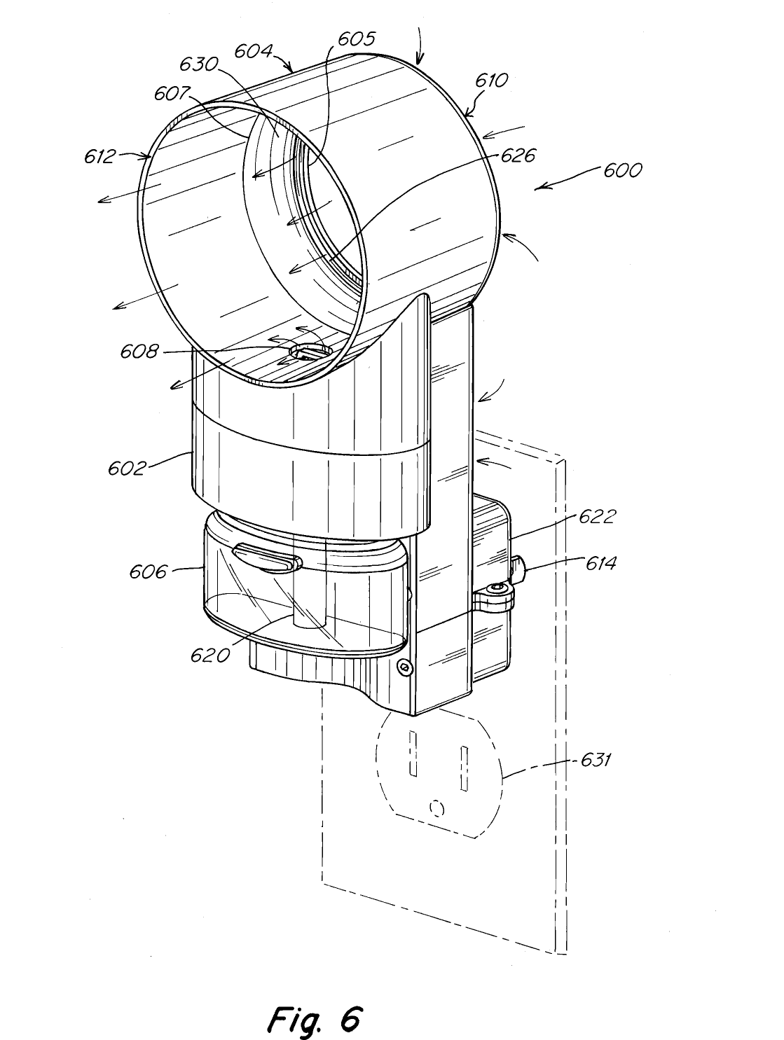

Referring to FIGS. 6-10, another embodiment of the formula dispersing apparatus 600 of the present invention is shown. Formula dispersing apparatus 600 includes a body 602 to which an airfoil 604 is attached. The taper of the ring airfoil 604 from thickest portion or leading edge 605 to the trailing edge 607 is constant and in the embodiment shown the angle of attack of the ring airfoil is 15 degrees. The portion of the airfoil 604 from the trailing edge 607 to the end of the airfoil on the pressure side 612 is a foil duct that is used to corral the air flow and move it across the porous media to entrain the formula in the air stream.

A removable formula reservoir 606 is connected to body 602 to supply formula which is discharged from the body 602 through opening 608 where it is entrained in the air supply travelling through the airfoil 604. Formula dispersing apparatus 600 is intended to be plugged into a wall socket 631 so that the airfoil 604 is spaced slightly from a wall 632 when the formula dispersing apparatus 600 is plugged into the wall socket 631. Air is drawn in from the suction side 610 of the airfoil 604 closest to the wall 630 and exits from the pressure end of the airfoil 604 on the side 612 furthest away from the wall 630.

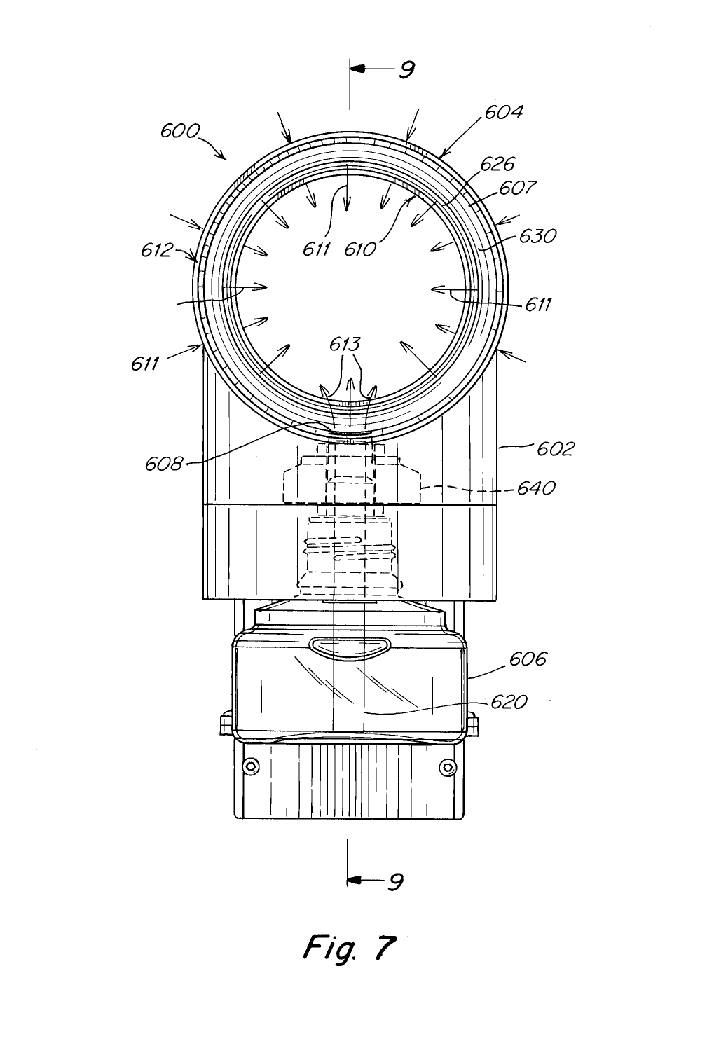

A front view of the formula dispersing apparatus 600 is shown in FIG. 7 in which the airflow 611 (which includes unmarked arrows around the perimeter as well) carries the formula emitting through opening 608 out of the airfoil 604.

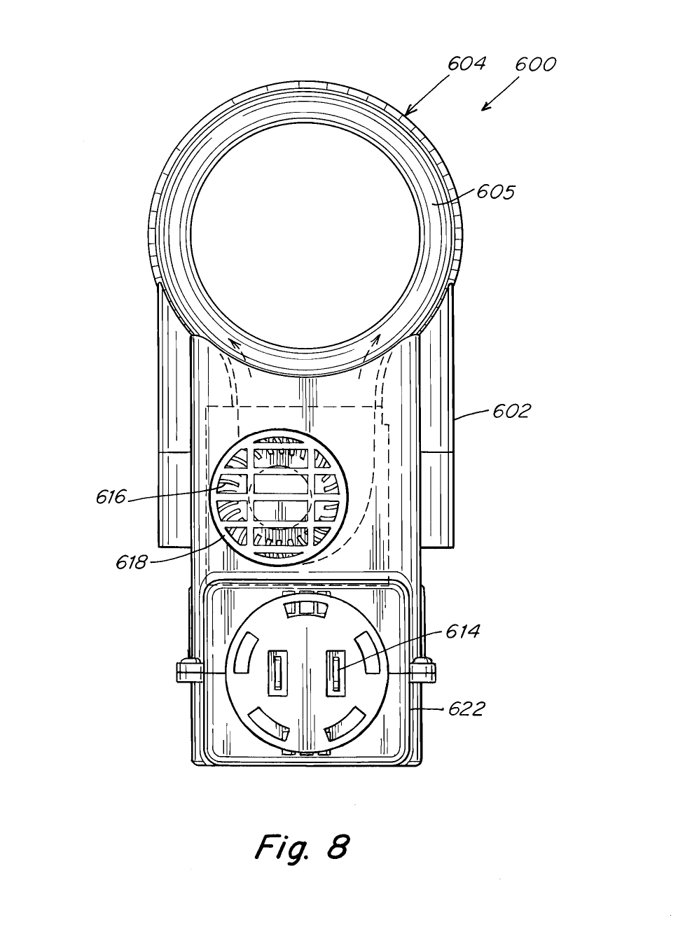

Referring now to FIG. 8, the rear side of the formula dispersing apparatus shown in FIGS. 6 and 7 is shown. AC power plug 614 is used to plug the formula dispersing apparatus 600 into an AC socket 631 to provide AC power to the formula dispersing apparatus 600. The power is used to run impeller 616 which is a fan housed behind grill 618. The impeller 616 blows air up into the ring airfoil 604 and then the air flowing out through the slot 626 in the ring airfoil 604, parallel to the ground and thus generating the Coand\check{a} effect. The rotation of the impeller 616 is axial to the ground.

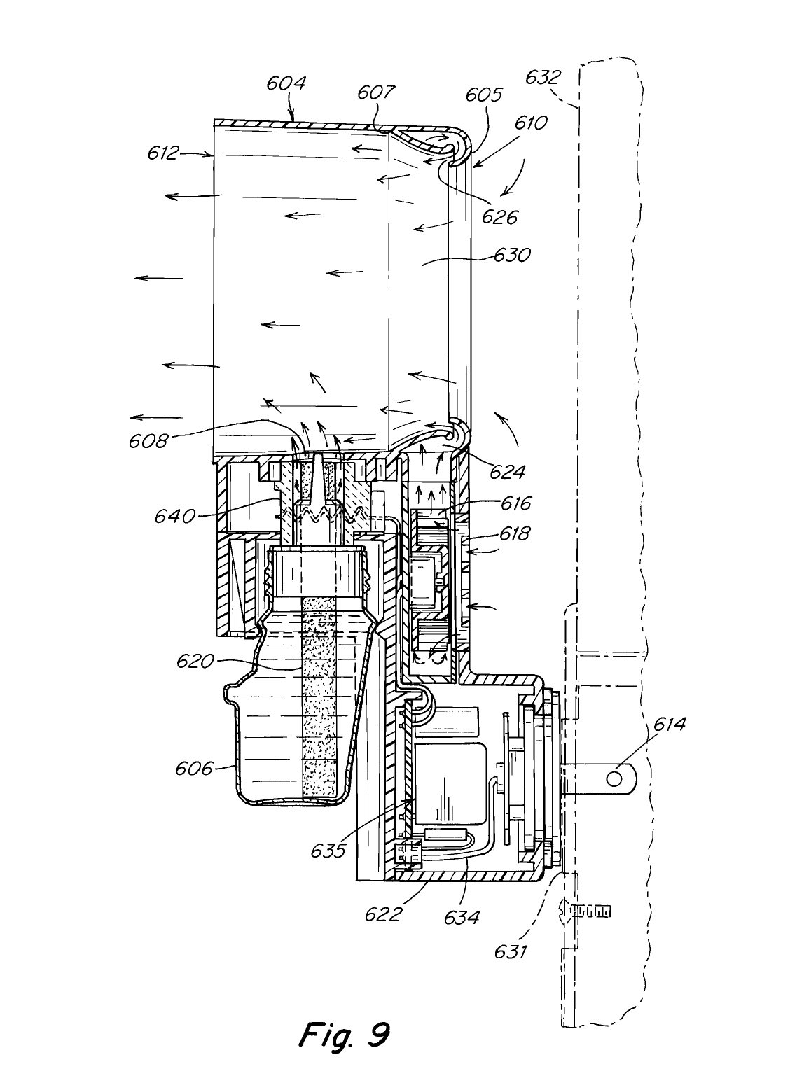

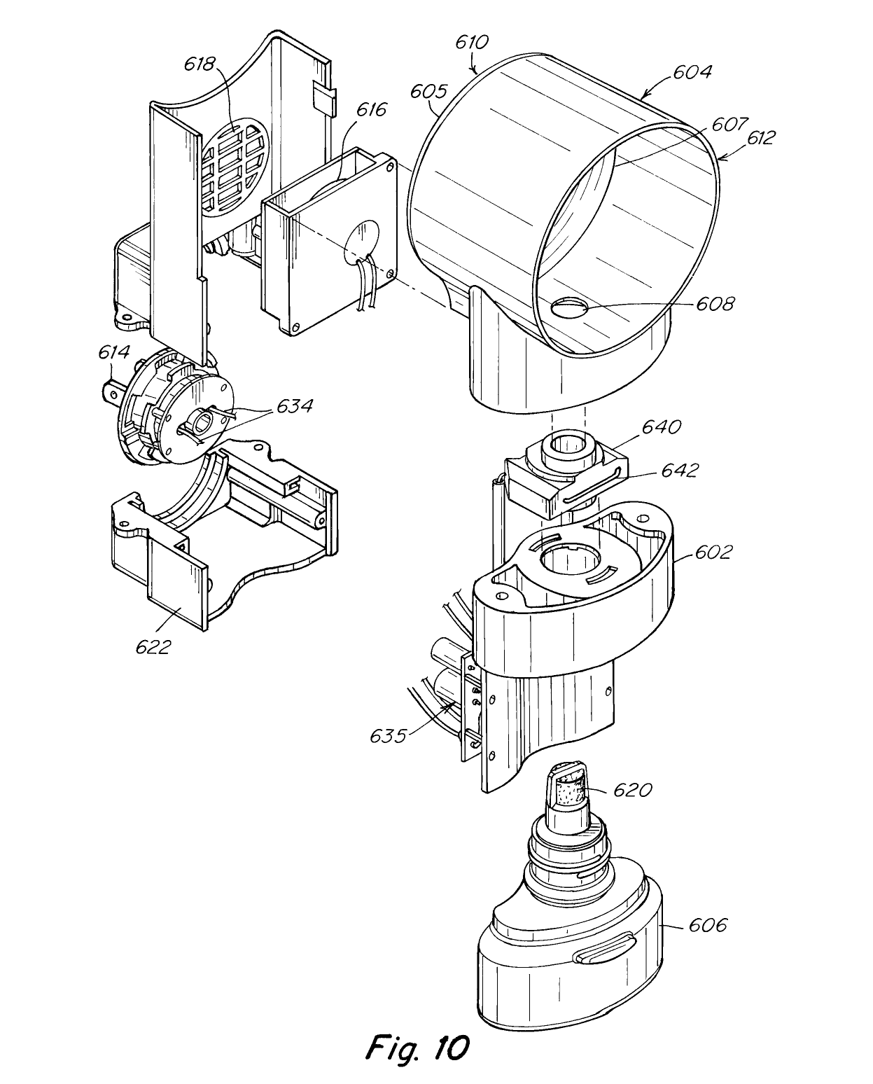

FIG. 9 shows a cross section and FIG. 10 shows an exploded view of the components of the airfoil 604 and the other internal components of the formula dispersing apparatus 600. The removable formula supply 606 which in this embodiment is a bottle includes a porous media (such as a cotton reed) 620 which extends into the bottle 606 and draws the liquid formula and carrier material by capillary action up to opening 608 where it becomes entrained in the air flow through air foil 604.

The plug 614 is supported in housing 622. The wires 634 from plug 614 are connected to a power supply 635 to adjust the voltage from line voltage (such as 120V AC or 22V AC) to one that will drive the fan (such as 5V DC or 12V DC). In addition, certain embodiments may include a means to alter or adjust the impeller 612 speed including but not limited to a rheostat, pulsed width modulation of the power, or discrete switched resistor values. Such control action can be done manually or via a preprogrammed or customizable programmed controller or microprocessor. As shown in FIG. 9 the impeller 616 draws air through grate 618 and forces it in an upward direction into a tunnel 624 before it is forced out through a slot 626 formed between the leading edge 605 of the ring airfoil 628 and the inner surface 630 of the ring airfoil 604. While in the embodiment shown in FIGS. 6-10 the carrier including the fragrance is emitted through opening 608, in other embodiments, the formula supply can be positioned (a) at a location relative to impeller 616 so that impeller 616 forces the carrier and formula into tunnel 624, (b) on the suction side 610 so that the carrier and formula is drawn into formula dispersing apparatus 600 through the suction side 610 or (c) between grate 618 and impeller 616 so that the impeller draws the formula into the apparatus 600 and discharges the formula into the tunnel 624.

In some embodiments the formula reservoir 606, in which the carrier and formula are incorporated, is heated. Whether heat is applied will depend on the volatility or vapor pressure of the carrier at ambient temperature. In the embodiment shown in FIGS. 6-10, a heater 640 is positioned near the upper portion of the porous media 620 where evaporation takes place. Heater 640 is a resistive heater with a resistor positioned on wire 642. In this embodiment it is preferred that the carrier and formula are heated to 120° F.-180° F. and more preferably 150° F. Other formulas would be heated to other temperature ranges. As is well known in the art a circuit board with a programmed integrated circuit would allow for a resistive heater to be both variable and/or intermittent based on either outside input or algorithms that are written to the programmed integrated circuit.

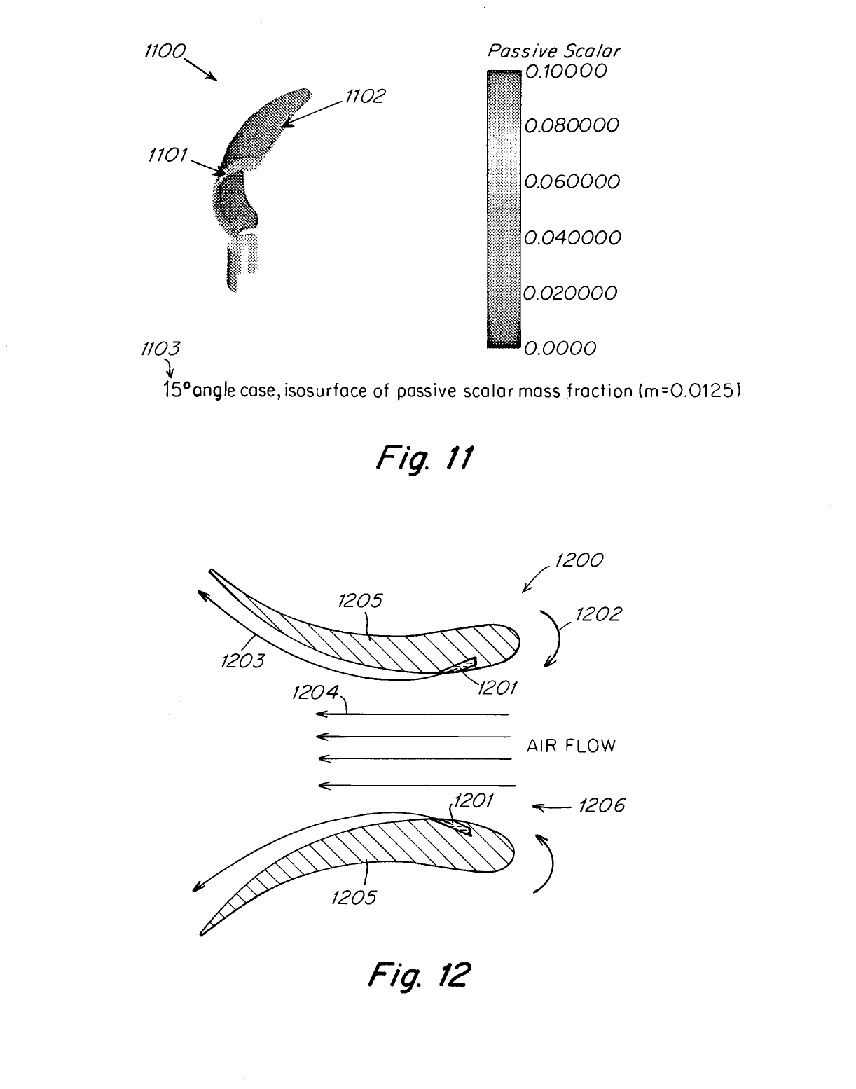

The configuration that is depicted in FIG. 11 depicts the computational fluid dynamics (CFD) analysis of a ring airfoil 1100 and the air flow downrange from the ring airfoil. The ring airfoil 1101 generates a multiplied airflow 1102. The angle of attack of the ring airfoil 1101 is modified to 15° 1103.

The air flow through the ring airfoil, utilizing the Coand\check{a} effect, is shown in FIG. 12. The airfoil configuration 1200 consists of the ring airfoil, shown in cross section 1205 with a slot 1201 running on the interior portion of the ring airfoil toward the intake 1206 of the ring airfoil. The air stream 1203 from the slot 1201 is flowing from the interior of the ring airfoil and is entrained with a formula. The air stream 1203 also allows for higher air flow 1204 due to the Coand\check{a} effect air stream 1203 keeping the air flow 1204 attached to the ring airfoil 1205 and thus enables higher efficiency flow through the ring airfoil 1201. This efficiency is accomplished by turning the flow 1202 through the intake 1206 of the ring airfoil 1205 and keeping the Coand\check{a} effect air stream 1203 attached to the ring airfoil 1205.

In FIG. 13, a 6412 airfoil 1300 is shown with a mean line 1301 and a trailing edge 1302.

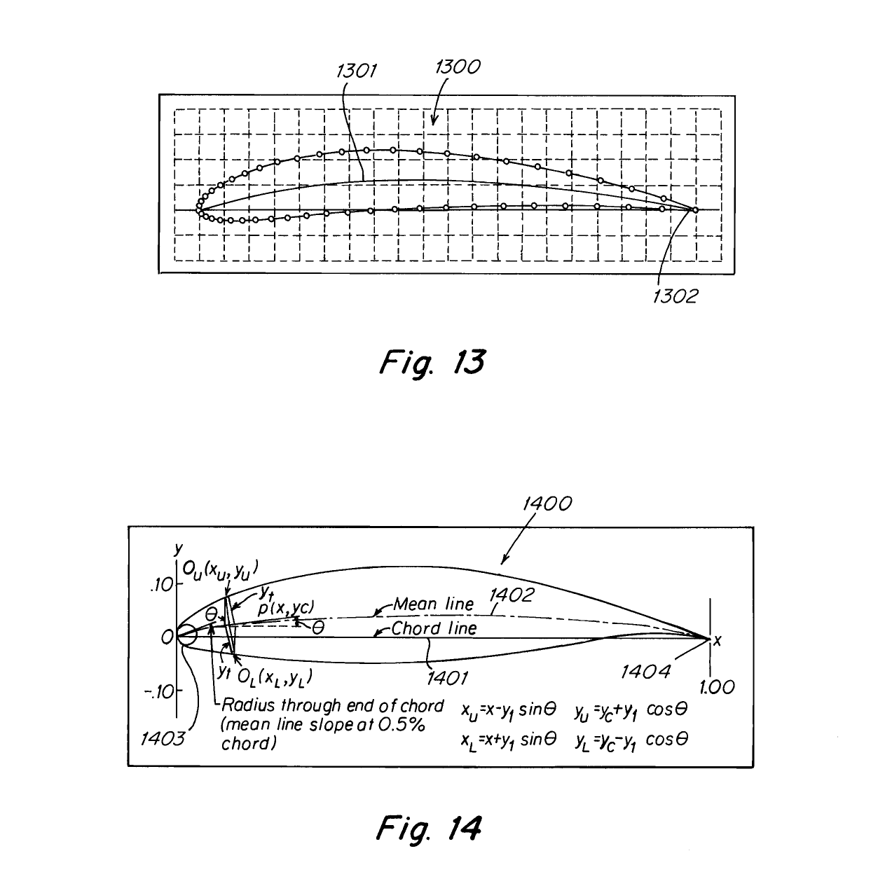

FIG. 14 shows a generic airfoil 1400 with a cord line 1401 and a mean line 1402. The cord line 1401 is a connection between the leading edge 1403 of the airfoil and the tip of the trailing edge 1404. The mean line 1402 bisects the airfoil from the leading edge 1403 to the trailing edge 1404.

While the invention has been described with respect to its preferred embodiments, various alterations and modifications will be apparent to those skilled in the art and all such alterations and modifications are intended to fall within the scope of the appended claims.

We claim:

1. A formula dispersing apparatus comprising:
a ring airfoil including a suction side and a pressure side, said airfoil including a tunnel and a slot said tunnel;
an impeller located in communication with said tunnel to force air into said tunnel;
a formula reservoir having an opening for releasing a material containing formula, said opening being positioned to enable said material containing formula released through said opening to pass through an outlet in said ring airfoil to be carried out the pressure side of said ring airfoil;
whereby formula is entrained into the air flow that is generated by the impeller and that flows through said slot in said ring airfoil.

2. The formula dispersing apparatus of claim 1 where the suction side of the airfoil is on the side of the ring airfoil that is closest to a wall when said formula dispersing apparatus is plugged into a wall.

3. The formula dispersing apparatus of claim 1 where the impeller axis is perpendicular to the flow of air over the ring airfoil.

4. The formula dispersing apparatus of claim 1 where access to said impeller is parallel to the flow of air over the ring airfoil.

5. The formula dispersing apparatus of claim 1 where the speed of the impeller is variable so as to increase or decrease the air flow.

6. The formula dispersing apparatus of claim 1 where formula is entrained into a flow stream and flowed out onto a ring airfoil utilizing the Coand\check{a} effect.

7. The formula dispersing apparatus of claim 1 where the taper of the ring airfoil from thickest portion to the trailing edge is constant around the ring airfoil.

8. The formula dispersing apparatus of claim 1 further comprising a heater for heating said formula.

9. The formula dispersing apparatus of claim 8 further comprising controls for intermittently applying the heat generated by said heater.

10. The formula dispersing apparatus of claim 8 further comprising controls for varying the temperature of the heat generated by said heater.

11. The formula dispersing apparatus of claim 1 wherein the material containing formula has a vapor pressure less than 200 kPa.

12. The formula dispersing apparatus of claim 1 wherein a porous media extends from said reservoir to draw the carrier containing formula out of said reservoir.
